(19) United States
(12) Patent Application Publication
(10) Pub. No.: US 2025/0127689 A1
(43) Pub. Date: Apr. 24, 2025

(54) METHOD OF STIMULATING DENTIN FORMATION WITH HYDROXY-SUBSTITUTED AZOBENZENE MATERIALS

(71) Applicant: The Regents of the University of Colorado, a body corporate, Denver, CO (US)

(72) Inventors: Devatha P. Nair, Denver (CO); Michael Schurr, Aurora, CO (US); Chaitanya Puranik, Aurora, CO (US)

(21) Appl. No.: 18/834,786

(22) PCT Filed: Jan. 10, 2023
(86) PCT No.: PCT/US2023/010455

§ 371 Date: Jul. 31, 2024

Related U.S. Application Data
(60) Provisional application No. 63/306,536, filed on Feb. 4, 2022.

(51) Int. Cl. A61K 6/887; A61K 6/52; A61K 6/71

(52) U.S. Cl. CPC A61K 6/887; A61K 6/52; A61K 6/71

(57) ABSTRACT

A method of stimulating dentin generation includes applying a polymerizable composition to exposed dental pulp in a dentate mammal or a dentate non-mammalian vertebrate; wherein the polymerizable composition comprises a hydroxy-substituted azobenzene group having the structure

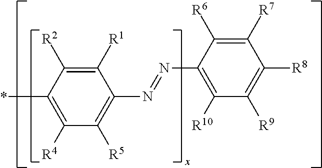

wherein x, R¹, R², R⁴, R⁵, R⁶, R⁷, R⁸, R⁹, and R¹⁰ are defined herein; and polymerizing the polymerizable composition; wherein the amount of the hydroxy-substituted azobenzene group applied to the exposed dental pulp is effective to promote dentin generation.

### CROSS-REFERENCE TO RELATED APPLICATION

This application claims the benefit of U.S. Provisional Application No. 63/306,536, filed Feb. 4, 2022, which is incorporated by reference herein in its entirety.

### STATEMENT REGARDING FEDERALLY SPONSORED RESEARCH & DEVELOPMENT

This invention was made with government support under Grant Number U01HL152405 awarded by the National Heart, Lung, and Blood Institute of the National Institutes of Health. The U.S. government has certain rights in the invention.

### BACKGROUND OF THE INVENTION

Various dental procedures, including root canals, involve the disruption of dentin and the exposure of dental pulp, thereby creating a need to regenerate dentin. The current standard of care in such procedures includes applying a mineral trioxide aggregate (MTA) root canal repair material to the exposed pulp. MTA materials are effective to stimulate generation of dentin, but they have various disadvantages, including the discoloration of repaired teeth, and the inability to be applied as a thin layer.

### BRIEF SUMMARY OF EMBODIMENTS OF THE INVENTION

One embodiment is a method of stimulating dentin formation, comprising: applying a polymerizable composition to exposed dental pulp in a dentate mammal or a dentate non-mammalian vertebrate; wherein the polymerizable composition comprises a hydroxy-substituted azobenzene group having the structure

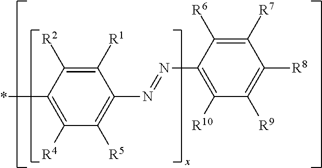

wherein x is 1, 2, 3, 4, 5, 6, 7, 8, 9, or 10; and each occurrence of R¹, R², R⁴, R⁵, R⁶, R⁷, R⁸, R⁹, and R¹⁰ is independently hydrogen, methyl, or hydroxyl, provided that at least one occurrence of R¹, R², R⁴, R⁵, R⁶, R⁷, R⁸, R⁹, and R¹⁰ is hydroxyl; and polymerizing the polymerizable composition; wherein the amount of the hydroxy-substituted azobenzene group applied to the exposed dental pulp is effective to promote dentin generation.

This and other embodiments are described in detail below.

### BRIEF DESCRIPTION OF THE DRAWINGS

FIG. 1 shows the structure of 2-acryloyloxy-2′-hydroxyazobenzene (Acrylated HydroxyAzobenzene, or AHA).

FIG. 2 is an optical micrograph of a hemotoxylin and eosin stained histological section of a murine maxillary first molar four weeks after treatment with a cured restorative comprising the polymerized residue of AHA; the black diamond indicates the site of pulp exposure, the black asterisk indicates the formation of dentin matrix (initial indication of formation of dentinal bridge), and the black arrows indicate vital pulp; the scale bar in the lower right corner corresponds to 100 micrometers.

FIG. 3 consists of five images (3A-3E) obtained by micro computed tomography (microCT).

FIG. 3A is a longitudinal section of a murine maxillary first molar harvested after creation of a cavity, but before treatment; the white diamond is situated just above an area of exposed pulp; the scale bar in the lower right corner corresponds to 100 micrometers.

FIG. 3B is a longitudinal section of a murine maxillary first molar harvested six weeks after a control dental procedure in which a cavity was created, SCOTCHBOND™ dental adhesive was applied to exposed dentin and cured, ProRoot™ MTA mineral trioxide aggregate root canal repair material was applied to exposed pulp, and FILTEK™ Supreme Ultra Universal Restorative was applied to the cured dental adhesive and the root canal repair material and cured; the white star indicates the position of cured restorative, which was removed prior to imaging; the area surrounded by a black dashed line indicates a pulp exposure site closed by a calcified dentinal bridge; the scale bar in the lower right corner corresponds to 100 micrometers.

FIG. 3C is a longitudinal section of a murine maxillary first molar harvested six weeks after a test dental procedure in which a cavity was created, SCOTCHBOND™ dental adhesive was applied to exposed dentin and cured, a solution of AHA was applied to exposed pulp and air-dried; and FILTEK™ Supreme Ultra Universal Restorative was applied to the cured dental adhesive and the air-dried AHA layer and cured; the white star indicates the position of cured restorative, which was removed prior to imaging; the area surrounded by a black dashed line indicates a pulp exposure site closed by a calcified dentinal bridge; the scale bar in the lower right corner corresponds to 100 micrometers.

FIG. 3D is a coronal section of the same murine maxillary first molar imaged in FIG. 3B; the area surrounded by black long-dash lines indicates an area of formation of a calcified dentinal bridge; the area surrounded by gray short-dash lines indicates an area of MTA placement above the calcified dentinal bridge; the area surrounded by white dashed lines indicates an area of the overlying cured restorative; the scale bar in the lower right corner corresponds to 100 micrometers.

FIG. 3E is a coronal section of the same murine maxillary first molar imaged in FIG. 3C; the area surrounded by black dashed lines indicates an area of formation of a calcified dentinal bridge; the area designated by a gray solid line indicates the area of AHA placement above the calcified dentinal bridge; the area surrounded by white dashed lines indicates an area of the overlying cured restorative; the scale bar in the lower right corner corresponds to 100 micrometers.

### DETAILED DESCRIPTION OF THE INVENTION

The present inventors have determined that dentin generation is stimulated by the application of a polymerizable composition to exposed dental pulp, wherein the polymerizable composition comprises a hydroxy-substituted azobenzene group having the structure specified below. The composition can be applied as a thin layer, and it is a potent stimulator of dentin generation. Thus, one embodiment is a method of stimulating dentin formation, comprising: applying a polymerizable composition to exposed dental pulp in a dentate mammal or a dentate non-mammalian vertebrate; wherein the polymerizable composition comprises a hydroxy-substituted azobenzene group having the structure

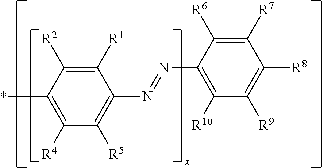

wherein x is 1, 2, 3, 4, 5, 6, 7, 8, 9, or 10; and each occurrence of R¹, R², R⁴, R⁵, R⁶, R⁷, R⁸, R⁹, and R¹⁰ is independently hydrogen, methyl, or hydroxyl, provided that at least one occurrence of R¹, R², R⁴, R⁵, R⁶, R⁷, R⁸, R⁹, and R¹⁰ is hydroxyl; and polymerizing the polymerizable composition; wherein the amount of the polymerizable composition to applied to the exposed dental pulp is effective to promote dentin generation.

The method of stimulating dentin formation is applicable to dentate mammals and dentate non-mammalian vertebrates. Included among these are mammalian and non-mammalian homodonts, heterodonts, diphyodonts, polyphyodonts, and thecodonts. In some embodiments, the method is applicable to humans and companion animals. In some embodiments, the method is applicable to humans.

The method comprises applying a polymerizable composition to exposed dental pulp in a dentate mammal or a dentate non-mammalian vertebrate. There is no particular limitation on the method by which the polymerizable composition can be applied to the exposed dental pulp. Suitable application methods include, for example, application via syringe, application via brush, application via spatula, and combinations thereof.

The polymerizable composition comprises a hydroxy-substituted azobenzene group having the structure

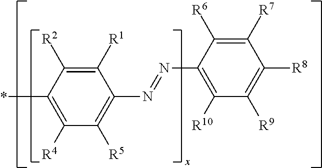

wherein x is 1, 2, 3, 4, 5, 6, 7, 8, 9, or 10; and each occurrence of R¹, R², R⁴, R⁵, R⁶, R⁷, R⁸, R⁹, and R¹⁰ is independently hydrogen, methyl, or hydroxyl, provided that at least one occurrence of R¹, R², R⁴, R⁵, R⁶, R⁷, R⁸, R⁹, and R¹⁰ is hydroxyl. In some embodiments, x is 1, 2, or 3. In some embodiments, x is 1 or 2. In some embodiments, x is 1. In some embodiments, one or two occurrences of R¹, R², R⁴, R⁵, R⁶, R⁷, R⁸, R⁹, and R¹⁰ are hydroxyl. In some embodiments, one occurrence of R¹, R², R⁴, R⁵, R⁶, R⁷, R⁸, R⁹, and R¹⁰ is hydroxyl. In some embodiments, one or two occurrences of R¹, R⁵, R⁶, and R¹⁰ are hydroxyl. In some embodiments, one occurrence of R¹, R⁵, R⁶, and R¹⁰ is hydroxyl. In some embodiments, x is 1, and one or two occurrences of R¹, R⁵, R⁶, and R¹⁰ are hydroxyl. In some embodiments, the hydroxy-substituted azobenzene group has the structure

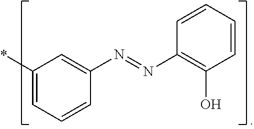

The amount of the hydroxy-substituted azobenzene group applied to the exposed dental pulp is effective to promote dentin generation. The effective dose of the hydroxy-substituted azobenzene group can be expressed various ways. In some embodiments, the polymerizable composition is applied to exposed dental pulp in an amount effective to provide 0.5 to 50 micrograms of the hydroxy-substituted azobenzene group. Within this range, the dose can be 1 to 40 micrograms of the hydroxy-substituted azobenzene group, or 2 to 15 micrograms of the hydroxy-substituted azobenzene group. In other embodiments, the polymerizable composition is applied to exposed dental pulp in an amount effective to provide 5 to 500 micrograms of the hydroxy-substituted azobenzene group per millimeter-squared of the exposed dental pulp. Within this range, the dose can be 10 to 250 micrograms of the hydroxy-substituted azobenzene group per millimeter-squared of the exposed dental pulp. In still other embodiments, the polymerizable composition is applied to exposed dental pulp in an amount effective to provide 20 to 3000 micrograms of the hydroxy-substituted azobenzene group per kilogram of body weight of the dentate mammal or a dentate non-mammalian vertebrate. Within this range, the effective dose can be 30 to 2500 micrograms of the hydroxy-substituted azobenzene group per kilogram of body weight of the dentate mammal or a dentate non-mammalian vertebrate, or 50 to 2000 micrograms of the hydroxy-substituted azobenzene group per kilogram of body weight of the dentate mammal or a dentate non-mammalian vertebrate.

In some embodiments, the hydroxy-substituted azobenzene group is part of a polymerizable azobenzene monomer having the structure

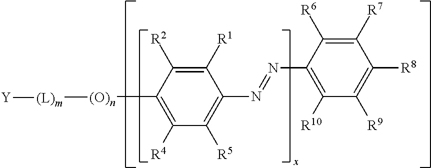

wherein m is zero or 1, and n is zero or 1, provided that if m is zero, then n is zero; L is C₂-C₁₂ alkylene or C₆-C₁₂ arylene; and Y is
(meth)acryloyloxy (—O—C(O)—C(R¹¹)═CH₂, wherein R¹¹ is hydrogen or methyl), and x, R¹, R², R⁴, R⁵, R⁶, R⁷, R⁸, R⁹, and R¹⁰ are as most broadly defined above; or
(meth)acrylamido (—N(H)—C(O)—C(R¹¹)═CH₂, wherein R¹¹ is hydrogen or methyl), and x, R¹, R², R⁴, R⁵, R⁶, R⁷, R⁸, R⁹, and R¹⁰ are as defined above; or
isocyanato (—N═C═O), and x, R¹, R², R⁴, R⁵, R⁶, R⁷, R⁸, R⁹, and R¹⁰ are as most broadly defined above, except that one occurrence of R¹, R², R⁴, R⁵, R⁶, R⁷, R⁸, R⁹, and R¹⁰ is isocyanato; provided that the polymerizable composition further comprises a C₂-C₁₂ aliphatic diol, a C₂-C₁₂ aliphatic dithiol, or a combination thereof; or
isothiocyanato (—N═C═S), and x, R¹, R², R⁴, R⁵, R⁶, R⁷, R⁸, R⁹, and R¹⁰ are as most broadly defined above, except that one occurrence of R¹, R², R⁴, R⁵, R⁶, R⁷, R⁸, R⁹, and R¹⁰ is isothiocyanato; provided that the polymerizable composition further comprises a C₂-C₁₂ aliphatic diol, a C₂-C₁₂ aliphatic dithiol, or a combination thereof; or
carboxyl (—C(O)—OH), and x, R¹, R², R⁴, R⁵, R⁶, R⁷, R⁸, R⁹, and R¹⁰ are as most broadly defined above, except that one occurrence of R¹, R², R⁴, R⁵, R⁶, R⁷, R⁸, R⁹, and R¹⁰ is carboxyl; provided that the polymerizable composition further comprises a C₂-C₁₂ aliphatic diol, a C₂-C₁₂ aliphatic diamine, or a combination thereof; or
thiol (—SH), and x, R¹, R², R⁴, R⁵, R⁶, R⁷, R⁸, R⁹, and R¹⁰ are as most broadly defined above, except that one occurrence of R¹, R², R⁴, R⁵, R⁶, R⁷, R⁸, R⁹, and R¹⁰ is thiol; provided that the polymerizable composition further comprises a C₂-C₁₂ aliphatic diisocyanate, a C₂-C₁₂ aliphatic diisothiocyanate, or a combination thereof; or
hydroxyl (—OH), and x, R¹, R², R⁴, R⁵, R⁶, R⁷, R⁸, R⁹, and R¹⁰ are as most broadly defined above, except that one occurrence of R¹, R², R⁴, R⁵, R⁶, R⁷, R⁸, R⁹, and R¹⁰ is hydroxyl; provided that the polymerizable composition further comprises a C₂-C₁₂ aliphatic diisocyanate, a C₂-C₁₂ aliphatic diisothiocyanate, or a combination thereof.

In some embodiments, Y is (meth)acryloyloxy; L is C₂-C₁₂ alkylene or C₆-C₁₂ arylene; and x, R¹, R², R⁴, R⁵, R⁶, R⁷, R⁸, R⁹, and R¹⁰ are as most broadly defined above.

In some embodiments, Y is (meth)acrylamido; L is C₂-C₁₂ alkylene or C₆-C₁₂ arylene; and x, R¹, R², R⁴, R⁵, R⁶, R⁷, R⁸, R⁹, and R¹⁰ are as most broadly defined above.

In other embodiments, Y is isocyanato (—N═C═O); L is C₂-C₁₂ alkylene or C₆-C₁₂ arylene; and x, R¹, R², R⁴, R⁵, R⁶, R⁷, R⁸, R⁹, and R¹⁰ are as most broadly defined above, except that one occurrence of R¹, R², R⁴, R⁵, R⁶, R⁷, R⁸, R⁹, and R¹⁰ is isocyanato, provided that the polymerizable composition further comprises a C₂-C₁₂ aliphatic diol, a C₂-C₁₂ aliphatic dithiol, or a combination thereof. The resulting diisocyanate is capable of reacting with the C₂-C₁₂ aliphatic diol, C₂-C₁₂ aliphatic dithiol, or combination thereof to form a polymer comprising the hydroxy-substituted azobenzene group.

In other embodiments, Y is isothiocyanato (—N═C═S); m is zero or 1, and n is zero or 1, provided that if m is zero, then n is zero; L is C₂-C₁₂ alkylene or C₆-C₁₂ arylene; and x, R¹, R², R⁴, R⁵, R⁶, R⁷, R⁸, R⁹, and R¹⁰ are as most broadly defined above, except that one occurrence of R¹, R², R⁴, R⁵, R⁶, R⁷, R⁸, R⁹, and R¹⁰ is isothiocyanato, provided that the polymerizable composition further comprises a C₂-C₁₂ aliphatic diol, a C₂-C₁₂ aliphatic dithiol, or a combination thereof. The resulting diisothiocyanate is capable of reacting with the C₂-C₁₂ aliphatic diol, C₂-C₁₂ aliphatic dithiol, or combination thereof to form a polymer comprising the hydroxy-substituted azobenzene group.

In other embodiments, Y is carboxyl (—C(O)—OH), and x, R¹, R², R⁴, R⁵, R⁶, R⁷, R⁸, R⁹, and R¹⁰ are as most broadly defined above, except that one occurrence of R¹, R², R⁴, R⁵, R⁶, R⁷, R⁸, R⁹, and R¹⁰ is carboxyl; provided that the polymerizable composition further comprises a C₂-C₁₂ aliphatic diol, a C₂-C₁₂ aliphatic diamine, or a combination thereof. The resulting dicarboxylic acid is capable of reacting with the C₂-C₁₂ aliphatic diol, C₂-C₁₂ aliphatic dithiol, or combination thereof to form a polymer comprising the hydroxy-substituted azobenzene group.

In other embodiments, Y is thiol (—SH), and x, R¹, R², R⁴, R⁵, R⁶, R⁷, R⁸, R⁹, and R¹⁰ are as most broadly defined above, except that one occurrence of R¹, R², R⁴, R⁵, R⁶, R⁷, R⁸, R⁹, and R¹⁰ is thiol; provided that the polymerizable composition further comprises a C₂-C₁₂ aliphatic diisocyanate, a C₂-C₁₂ aliphatic diisothiocyanate, or a combination thereof. The resulting dithiol is capable of reacting with the C₂-C₁₂ aliphatic diisocyanate, C₂-C₁₂ aliphatic diisothiocyanate, or combination thereof to form a polymer comprising the hydroxy-substituted azobenzene group.

In other embodiments, Y is hydroxyl (—OH), and x, R¹, R², R⁴, R⁵, R⁶, R⁷, R⁸, R⁹, and R¹⁰ are as most broadly defined above, except that one occurrence of R¹, R², R⁴, R⁵, R⁶, R⁷, R⁸, R⁹, and R¹⁰ is hydroxyl; provided that the polymerizable composition further comprises a C₂-C₁₂ aliphatic diisocyanate, a C₂-C₁₂ aliphatic diisothiocyanate, or a combination thereof. The resulting diol is capable of reacting with the C₂-C₁₂ aliphatic diisocyanate, C₂-C₁₂ aliphatic diisothiocyanate, or combination thereof to form a polymer comprising the hydroxy-substituted azobenzene group.

In some embodiments of the polymerizable azobenzene monomer, Y is (meth)acryloyloxy; m and n are zero; x is 1; and 1 or 2 occurrences of R¹, R⁵, R⁶, and R¹⁰ are hydroxyl.

In other embodiments, the polymerizable azobenzene monomer has the structure

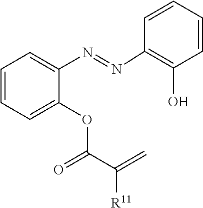

wherein R¹¹ is hydrogen or methyl.

When the hydroxy-substituted azobenzene group is part of a polymerizable azobenzene monomer, the polymerizable composition can consist of the polymerizable azobenzene monomer.

Alternatively, the polymerizable composition can comprise the polymerizable azobenzene monomer and further comprise a solvent selected from the group consisting of C₁-C₆ alkanols (including ethanol), C₃-C₆ ketones (including acetone and methyl ethyl ketone), and combinations thereof (including the combination of ethanol and acetone). When the polymerizable composition comprises a polymerizable azobenzene monomer and a solvent, the polymerizable composition can comprise 0.05 to 10 weight percent of the polymerizable azobenzene monomer, and 90 to 99.95 weight percent of the solvent. Within these ranges, the polymerizable composition can comprise 0.1 to 5 weight percent of the polymerizable azobenzene monomer, and 95 to 99.95weight percent of the solvent, or 0.15 to 2.5 weight percent of the polymerizable azobenzene monomer, and 97.5 to 99.85weight percent of the solvent.

Alternatively, the polymerizable composition can comprise the polymerizable azobenzene monomer and further comprise a copolymerizable monomer. For example, when the polymerizable azobenzene monomer comprises a (meth)acryloyloxy group or a (meth)acrylamido group, the copolymerizable monomer can be a monofunctional (meth)acrylate (such as a C₁-C₆-alkyl (meth)acrylate, a hydroxy-C₁-C₆-alkyl (meth)acrylate, or a combination thereof), a difunctional (meth)acrylate (such as bisphenol A diglycidyl ether di(meth)acrylate, bisphenol A polyethylene glycol diether di(meth)acrylate, diurethane di(meth)acrylate, propylene glycol di(meth)acrylate, diethylene glycol di(meth)acrylate, triethylene glycol di(meth)acrylate, or a combination thereof), or a combination thereof.

Alternatively, the polymerizable composition can comprise the polymerizable azobenzene monomer and further comprise a solvent and a copolymerizable monomer.

In some embodiments, the hydroxy-substituted azobenzene group is a pendant group of a polymer, and the polymerizable composition further comprises a curable monomer. Specifically, the polymerizable composition further comprises a curable monomer; and the hydroxy-substituted azobenzene group is a pendant group of a polymer comprising the residue of a polymerizable azobenzene monomer having the structure

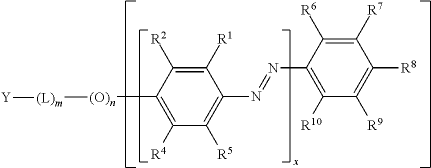

wherein m is zero or 1, and n is zero or 1, provided that if m is zero, then n is zero; L is C₂-C₁₂ alkylene or C₆-C₁₂ arylene; and Y is
(meth)acryloyloxy, and x, R¹, R², R⁴, R⁵, R⁶, R⁷, R⁸, R⁹, and R¹⁰ are defined as in claim 1; or
(meth)acrylamido, and x, R¹, R², R⁴, R⁵, R⁶, R⁷, R⁸, R⁹, and R¹⁰ are defined as in claim 1; or
isocyanato, and x, R¹, R², R⁴, R⁵, R⁶, R⁷, R⁸, R⁹, and R¹⁰ are defined as in claim 1, except that one occurrence of R¹, R², R⁴, R⁵, R⁶, R⁷, R⁸, R⁹, and R¹⁰ is isocyanato; provided that the polymerizable composition comprises further comprises a C₂-C₁₂ aliphatic diol, a C₂-C₁₂ aliphatic dithiol, or a combination thereof; or
isothiocyanato, and x, R¹, R², R⁴, R⁵, R⁶, R⁷, R⁸, R⁹, and R¹⁰ are defined as in claim 1, except that one occurrence of R¹, R², R⁴, R⁵, R⁶, R⁷, R⁸, R⁹, and R¹⁰ is isothiocyanato; provided that the polymerizable composition comprises further comprises a C₂-C₁₂ aliphatic diol, a C₂-C₁₂ aliphatic dithiol, or a combination thereof; or
carboxyl, and x, R¹, R², R⁴, R⁵, R⁶, R⁷, R⁸, R⁹, and R¹⁰ are defined as in claim 1, except that one occurrence of R¹, R², R⁴, R⁵, R⁶, R⁷, R⁸, R⁹, and R¹⁰ is carboxyl; provided that the polymerizable composition comprises further comprises a C₂-C₁₂ aliphatic diol, a C₂-C₁₂ aliphatic diamine, or a combination thereof; or
thiol, and x, R¹, R², R⁴, R⁵, R⁶, R⁷, R⁸, R⁹, and R¹⁰ are defined as in claim 1, except that one occurrence of R¹, R², R⁴, R⁵, R⁶, R⁷, R⁸, R⁹, and R¹⁰ is thiol; provided that the polymerizable composition comprises further comprises a C₂-C₁₂ aliphatic diisocyanate, a C₂-C₁₂ aliphatic diisothiocyanate, or a combination thereof; or
hydroxyl, and x, R¹, R², R⁴, R⁵, R⁶, R⁷, R⁸, R⁹, and R¹⁰ are defined as in claim 1, except that one occurrence of R¹, R², R⁴, R⁵, R⁶, R⁷, R⁸, R⁹, and R¹⁰ is hydroxyl; provided that the polymerizable composition comprises further comprises a C₂-C₁₂ aliphatic diisocyanate, a C₂-C₁₂ aliphatic diisothiocyanate, or a combination thereof.

In these embodiments, the curable monomer can be, for example, a monofunctional (meth)acrylate (such as a C₁-C₆-alkyl (meth)acrylate, a hydroxy-C₁-C₆-alkyl (meth)acrylate, or a combination thereof), a difunctional (meth)acrylate (such as bisphenol A diglycidyl ether di(meth)acrylate, bisphenol A polyethylene glycol diether di(meth)acrylate, diurethane di(meth)acrylate, propylene glycol di(meth)acrylate, diethylene glycol di(meth)acrylate, triethylene glycol di(meth)acrylate, or a combination thereof), or a combination thereof.

In a subset of these embodiments, Y is (meth)acryloyloxy; m and n are zero; x is 1; and 1 or 2 occurrences of R¹, R⁵, R⁶, and R¹⁰ are hydroxyl.

In a narrower subset of these embodiments, the polymerizable azobenzene monomer has the structure

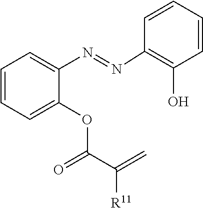

wherein R¹¹ is hydrogen or methyl.

In addition to the applying a polymerizable composition to exposed dental pulp, the method comprises the polymerizable composition. Methods of polymerizing a polymerizable composition are known in the art. For example, the polymerizing can be effected via thermal, self-curing, or chemical means, or a combination thereof. Thermal polymerization generally employs heat in combination with at least one thermal initiator, producing radicals that polymerize the composition. Examples of thermal initiators include peroxides, such as benzoyl peroxide. Self-curing polymerization generally employs at least two initiators, combined just prior to polymerization, that react to form radicals that polymerize the composition. Examples of self-curing initiators include the combination of a peroxide, such as benzoyl peroxide, and an amine, such as N,N-dimethyl-para-toluidine. And chemical polymerization generally employs ultraviolet or visible irradiation, optionally in the presence of at least one initiator, producing radicals that polymerize the composition. Examples of chemical initiators include ultraviolet and/or visible light (e.g., light have a wavelength of 400 to 500 nanometers) in combination with a quinone, such as camphorquinone, and an amine, such as ethyl-4-(dimethylamino)benzoate. A very specific example of a polymerizable composition comprises a (meth)acrylate-substituted polymerizable azobenzene monomer in combination with 400-500 nanometer light, benzoyl peroxide at 2 weight percent, N,N-dimethyl-para-toluidine at 1 weight percent, camphorquinone at 1 weight percent, and ethyl-4-(dimethylamino)benzoate at 1 weight percent, wherein the weight percents of all curing agents are based on the weight of the (meth)acrylate-substituted polymerizable azobenzene monomer.

In a very specific embodiment of the method, the dentate mammal or dentate non-mammalian vertebrate comprises exposed dentin in addition to exposed pulp; the method further comprises applying a dental adhesive to the exposed dentin and curing the dental adhesive, prior to the applying a polymerizable composition to exposed dental pulp; the method further comprises applying the polymerizable composition to the cured dental adhesive simultaneously with the applying the polymerizable composition to the exposed dental pulp, and, optionally, air-drying and/or polymerizing the polymerizable composition; the method further comprises applying a restorative composition to the cured dental adhesive and the (optionally air-dried and/or at least partially cured) polymerizable composition, and polymerizing the restorative composition (the polymerizable composition can be polymerized separately from and/or together with the restorative composition); wherein the restorative composition comprises 50 to 95 weight percent of a particulate inorganic (mineral or ceramic) material, and 5 to 50 weight percent of a di(meth)acrylate monomer (describe), based on the total weight of the restorative composition; the polymerizable composition further comprises a solvent selected from the group consisting of C₁-C₆ alkanols (ethanol), C₃-C₆ ketones (acetone, MEK; acetone), and combinations thereof (for example, ethanol and acetone); and the hydroxy-substituted azobenzene group is a pendant group of a polymerizable azobenzene monomer having the structure

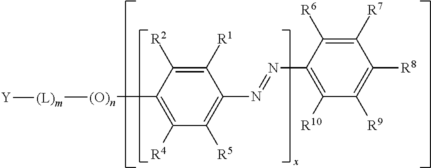

wherein m is zero or 1, and n is zero or 1, provided that if m is zero, then n is zero; L is C₂-C₁₂ alkylene or C₆-C₁₂ arylene; and Y is (meth)acryloyloxy, and x, R¹, R², R⁴, R⁵, R⁶, R⁷, R⁸, R⁹, and R¹⁰ are defined as in claim 1; or (meth)acrylamido, and x, R¹, R², R⁴, R⁵, R⁶, R⁷, R⁸, R⁹, and R¹⁰ are as defined most broadly above.

In some variations of this very specific embodiment, the polymerizable composition comprises, based on the weight of the polymerizable composition, 0.1 to 5 weight percent of the polymerizable azobenzene monomer, and 10 to 99.9 weight percent of the solvent, or 0.15 to 2.5 weight percent of the polymerizable azobenzene monomer, and 97.5 to 99.85 weight percent of the solvent. The polymerizable composition can, optionally, further comprise 5 to 89.9 weight percent of a (meth)acrylate monomer copolymerizable with the polymerizable monomer, based on the weight of the polymerizable composition. Such copolymerizable (meth)acrylate monomers include, for example, for example, a monofunctional (meth)acrylate (such as a C₁-C₆-alkyl (meth)acrylate, a hydroxy-C₁-C₆-alkyl (meth)acrylate, or a combination thereof), a difunctional (meth)acrylate (such as bisphenol A diglycidyl ether di(meth)acrylate, bisphenol A polyethylene glycol diether di(meth)acrylate, diurethane di(meth)acrylate, propylene glycol di(meth)acrylate, diethylene glycol di(meth)acrylate, triethylene glycol di(meth)acrylate, or a combination thereof), or a combination thereof.

In another very specific embodiment of the method, the dentate mammal or dentate non-mammalian vertebrate comprises exposed dentin in addition to exposed pulp; the method further comprises applying a dental adhesive to the exposed dentin and curing the dental adhesive, after the applying a polymerizable composition to exposed dental pulp; the method further comprises, optionally, air-drying and/or polymerizing the polymerizable composition prior to the applying a dental adhesive to the exposed dentin and curing the dental adhesive; the method further comprises applying a restorative composition to the cured dental adhesive and the (optionally air-dried and/or at least partially cured) polymerizable composition, and polymerizing the restorative composition (the polymerizable composition can be polymerized separately from and/or together with the restorative composition); wherein the restorative composition comprises 50 to 95 weight percent of a particulate inorganic (mineral or ceramic) material, and 5 to 50 weight percent of a di(meth)acrylate monomer (describe), based on the total weight of the restorative composition; the polymerizable composition further comprises a solvent selected from the group consisting of C₁-C₆ alkanols (ethanol), C₃-C₆ ketones (acetone, MEK; acetone), and combinations thereof (for example, ethanol and acetone); and the hydroxy-substituted azobenzene group is a pendant group of a polymerizable azobenzene monomer having the structure

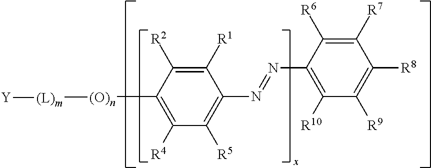

wherein m is zero or 1, and n is zero or 1, provided that if m is zero, then n is zero; L is C₂-C₁₂ alkylene or C₆-C₁₂ arylene; and Y is (meth)acryloyloxy, and x, R¹, R², R⁴, R⁵, R⁶, R⁷, R⁸, R⁹, and R¹⁰ are defined as in claim 1; or (meth)acrylamido, and x, R¹, R², R⁴, R⁵, R⁶, R⁷, R⁸, R⁹, and R¹⁰ are as defined most broadly above.

In some variations of this very specific embodiment, the polymerizable composition comprises, based on the weight of the polymerizable composition, 0.1 to 5 weight percent of the polymerizable azobenzene monomer, and 10 to 99.9 weight percent of the solvent, or 0.15 to 2.5 weight percent of the polymerizable azobenzene monomer, and 97.5 to 99.85 weight percent of the solvent. The polymerizable composition can, optionally, further comprise 5 to 89.9 weight percent of a (meth)acrylate monomer copolymerizable with the polymerizable monomer, based on the weight of the polymerizable composition. Such copolymerizable (meth)acrylate monomers include, for example, for example, a monofunctional (meth)acrylate (such as a C₁-C₆-alkyl (meth)acrylate, a hydroxy-C₁-C₆-alkyl (meth)acrylate, or a combination thereof), a difunctional (meth)acrylate (such as bisphenol A diglycidyl ether di(meth)acrylate, bisphenol A polyethylene glycol diether di(meth)acrylate, diurethane di(meth)acrylate, propylene glycol di(meth)acrylate, diethylene glycol di(meth)acrylate, triethylene glycol di(meth)acrylate, or a combination thereof), or a combination thereof.

In another very specific embodiment of the method, the dentate mammal or dentate non-mammalian vertebrate comprises exposed dentin in addition to exposed pulp; the method further comprises applying a dental adhesive to the exposed dentin and curing the dental adhesive prior to the applying a polymerizable composition to exposed dental pulp; the method further comprises applying the polymerizable composition to the cured dental adhesive simultaneously with the applying the polymerizable composition to the exposed dental pulp; and the hydroxy-substituted azobenzene group is a pendant group of a polymerizable azobenzene monomer having the structure

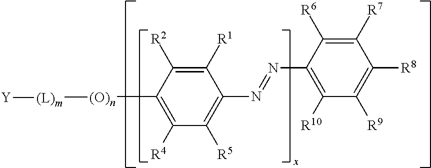

wherein m is zero or 1, and n is zero or 1, provided that if m is zero, then n is zero; L is C₂-C₁₂ alkylene or C₆-C₁₂ arylene; and Y is (meth)acryloyloxy, and x, R¹, R², R⁴, R⁵, R⁶, R⁷, R⁸, R⁹, and R¹⁰ are defined as in claim 1; or (meth)acrylamido, and x, R¹, R², R⁴, R⁵, R⁶, R⁷, R⁸, R⁹, and R¹⁰ are defined as in claim 1; and the polymerizable composition is a restorative composition comprising the polymerizable azobenzene monomer, a particulate inorganic filler (mineral or ceramic material), and a (meth)acrylate monomer copolymerizable with the polymerizable monomer.

In some embodiments of this method, the (meth)acrylate monomer copolymerizable with the polymerizable monomer comprises a monofunctional (meth)acrylate (such as a C₁-C₆-alkyl (meth)acrylate, a hydroxy-C₁-C₆-alkyl (meth)acrylate, or a combination thereof), a difunctional (meth)acrylate (such as bisphenol A diglycidyl ether di(meth)acrylate, bisphenol A polyethylene glycol diether di(meth)acrylate, diurethane di(meth)acrylate, propylene glycol di(meth)acrylate, diethylene glycol di(meth)acrylate, triethylene glycol di(meth)acrylate, or a combination thereof), or a combination thereof.

In some embodiments of this method, the restorative composition comprises, based on the total weight of the restorative composition, 0.1 to 5 weight percent of the polymerizable azobenzene monomer, 50 to 94.9 weight percent of the particulate inorganic material, and 5 to 50 weight percent of the (meth)acrylate monomer copolymerizable with the polymerizable azobenzene monomer. Within the above range of 0.1 to 5, the weight percent of the polymerizable azobenzene monomer can be 0.2 to 3 weight percent, or 0.3 to 2 weight percent.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

All ranges disclosed herein are inclusive of the endpoints, and the endpoints are independently combinable with each other. Each range disclosed herein constitutes a disclosure of any point or sub-range lying within the disclosed range.

The use of the terms “a” and “an” and “the” and similar referents in the context of describing the invention (especially in the context of the following claims) are to be construed to cover both the singular and the plural, unless otherwise indicated herein or clearly contradicted by context. Further, it should further be noted that the terms “first,” “second,” and the like herein do not denote any order, quantity, or importance, but rather are used to distinguish one element from another. The modifier “about” used in connection with a quantity is inclusive of the stated value and has the meaning dictated by the context (e.g., it includes the degree of error associated with measurement of the particular quantity).

The invention includes at least the following aspects.

Aspect 1: A method of stimulating dentin formation, comprising: applying a polymerizable composition to exposed dental pulp in a dentate mammal or a dentate non-mammalian vertebrate; wherein the polymerizable composition comprises a hydroxy-substituted azobenzene group having the structure

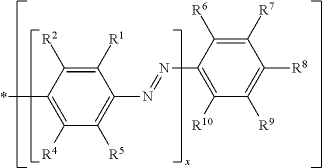

wherein x is 1, 2, 3, 4, 5, 6, 7, 8, 9, or 10; and each occurrence of R¹, R², R⁴, R⁵, R⁶, R⁷, R⁸, R⁹, and R¹⁰ is independently hydrogen, methyl, or hydroxyl, provided that at least one occurrence of R¹, R², R⁴, R⁵, R⁶, R⁷, R⁸, R⁹, and R¹⁰ is hydroxyl; and polymerizing the polymerizable composition; wherein the amount of the hydroxy-substituted azobenzene group applied to the exposed dental pulp is effective to promote dentin generation.

Aspect 2: The method of aspect 1, wherein, in the hydroxy-substituted azobenzene group, x is 1, and 1 or 2 occurrences of R¹, R⁵, R⁶, and R¹⁰ are hydroxyl.

Aspect 3: The method of aspect 1, wherein the hydroxy-substituted azobenzene group has the structure

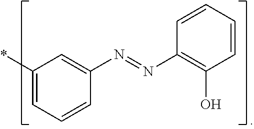

Aspect 4: The method of aspect 1, wherein the applying a polymerizable composition to exposed dental pulp comprises applying the polymerizable composition to exposed dental pulp in an amount effective to provide 0.5 to 50 micrograms of the hydroxy-substituted azobenzene group.

Aspect 5: The method of aspect 1, wherein the applying a polymerizable composition to exposed dental pulp comprises applying the polymerizable composition to exposed dental pulp in an amount effective to provide 5 to 500 micrograms of the hydroxy-substituted azobenzene group per millimeter-squared of the exposed dental pulp.

Aspect 6: The method of aspect 1, wherein the applying a polymerizable composition to exposed dental pulp comprises applying the polymerizable composition to exposed dental pulp in an amount effective to provide 20 to 3000 micrograms of the hydroxy-substituted azobenzene group per kilogram of body weight of the dentate mammal or a dentate non-mammalian vertebrate.

Aspect 7: The method of aspect 1, wherein the hydroxy-substituted azobenzene group is part of a polymerizable azobenzene monomer having the structure

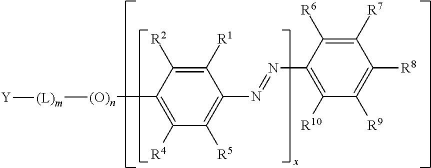

wherein m is zero or 1, and n is zero or 1, provided that if m is zero, then n is zero; L is C₂-C₁₂ alkylene or C₆-C₁₂ arylene; and Y is (meth)acryloyloxy, and x, R¹, R², R⁴, R⁵, R⁶, R⁷, R⁸, R⁹, and R¹⁰ are defined as in aspect 1; or (meth)acrylamido, and x, R¹, R², R⁴, R⁵, R⁶, R⁷, R⁸, R⁹, and R¹⁰ are defined as in aspect 1; or isocyanato, and x, R¹, R², R⁴, R⁵, R⁶, R⁷, R⁸, R⁹, and R¹⁰ are defined as in aspect 1, except that one occurrence of R¹, R², R⁴, R⁵, R⁶, R⁷, R⁸, R⁹, and R¹⁰ is isocyanato; provided that the polymerizable composition further comprises a C₂-C₁₂ aliphatic diol, a C₂-C₁₂ aliphatic dithiol, or a combination thereof; or isothiocyanato, and x, R¹, R², R⁴, R⁵, R⁶, R⁷, R⁸, R⁹, and R¹⁰ are defined as in aspect 1, except that one occurrence of R¹, R², R⁴, R⁵, R⁶, R⁷, R⁸, R⁹, and R¹⁰ is isothiocyanato; provided that the polymerizable composition further comprises a C₂-C₁₂ aliphatic diol, a C₂-C₁₂ aliphatic dithiol, or a combination thereof; or carboxyl, and x, R¹, R², R⁴, R⁵, R⁶, R⁷, R⁸, R⁹, and R¹⁰ are defined as in aspect 1, except that one occurrence of R¹, R², R⁴, R⁵, R⁶, R⁷, R⁸, R⁹, and R¹⁰ is carboxyl; provided that the polymerizable composition further comprises a C₂-C₁₂ aliphatic diol, a C₂-C₁₂ aliphatic diamine, or a combination thereof; or thiol, and x, R¹, R², R⁴, R⁵, R⁶, R⁷, R⁸, R⁹, and R¹⁰ are defined as in aspect 1, except that one occurrence of R¹, R², R⁴, R⁵, R⁶, R⁷, R⁸, R⁹, and R¹⁰ is thiol; provided that the polymerizable composition further comprises a C₂-C₁₂ aliphatic diisocyanate, a C₂-C₁₂ aliphatic diisothiocyanate, or a combination thereof; or hydroxyl, and x, R¹, R², R⁴, R⁵, R⁶, R⁷, R⁸, R⁹, and R¹⁰ are defined as in aspect 1, except that one occurrence of R¹, R², R⁴, R⁵, R⁶, R⁷, R⁸, R⁹, and R¹⁰ is hydroxyl; provided that the polymerizable composition further comprises a C₂-C₁₂ aliphatic diisocyanate, a C₂-C₁₂ aliphatic diisothiocyanate, or a combination thereof.

Aspect 8: The method of aspect 7, wherein, in the polymerizable azobenzene monomer, Y is (meth)acryloyloxy; m and n are zero; x is 1; and 1 or 2 occurrences of R¹, R⁵, R⁶, and R¹⁰ are hydroxyl.

Aspect 9: The method of aspect 7, wherein the polymerizable azobenzene monomer has the structure

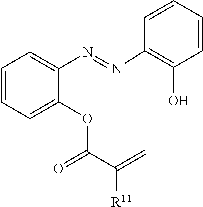

wherein R¹¹ is hydrogen or methyl.

Aspect 10: The method of aspect 1, wherein the polymerizable composition further comprises a curable monomer; and the hydroxy-substituted azobenzene group is a pendant group of a polymer comprising the residue of a polymerizable azobenzene monomer having the structure

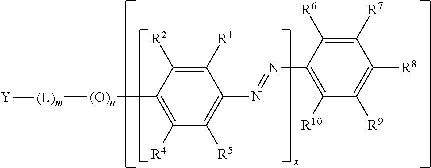

wherein m is zero or 1, and n is zero or 1, provided that if m is zero, then n is zero; L is C₂-C₁₂ alkylene or C₆-C₁₂ arylene; and Y is (meth)acryloyloxy, and x, R¹, R², R⁴, R⁵, R⁶, R⁷, R⁸, R⁹, and R¹⁰ are defined as in aspect 1; or (meth)acrylamido, and x, R¹, R², R⁴, R⁵, R⁶, R⁷, R⁸, R⁹, and R¹⁰ are defined as in aspect 1; or isocyanato, and x, R¹, R², R⁴, R⁵, R⁶, R⁷, R⁸, R⁹, and R¹⁰ are defined as in aspect 1, except that one occurrence of R¹, R², R⁴, R⁵, R⁶, R⁷, R⁸, R⁹, and R¹⁰ is isocyanato; provided that the polymerizable composition further comprises a C₂-C₁₂ aliphatic diol, a C₂-C₁₂ aliphatic dithiol, or a combination thereof; or isothiocyanato, and x, R¹, R², R⁴, R⁵, R⁶, R⁷, R⁸, R⁹, and R¹⁰ are defined as in aspect 1, except that one occurrence of R¹, R², R⁴, R⁵, R⁶, R⁷, R⁸, R⁹, and R¹⁰ is isothiocyanato; provided that the polymerizable composition further comprises a C₂-C₁₂ aliphatic diol, a C₂-C₁₂ aliphatic dithiol, or a combination thereof; or carboxyl, and x, R¹, R², R⁴, R⁵, R⁶, R⁷, R⁸, R⁹, and R¹⁰ are defined as in aspect 1, except that one occurrence of R¹, R², R⁴, R⁵, R⁶, R⁷, R⁸, R⁹, and R¹⁰ is carboxyl; provided that the polymerizable composition further comprises a C₂-C₁₂ aliphatic diol, a C₂-C₁₂ aliphatic diamine, or a combination thereof; or thiol, and x, R¹, R², R⁴, R⁵, R⁶, R⁷, R⁸, R⁹, and R¹⁰ are defined as in aspect 1, except that one occurrence of R¹, R², R⁴, R⁵, R⁶, R⁷, R⁸, R⁹, and R¹⁰ is thiol; provided that the polymerizable composition further comprises a C₂-C₁₂ aliphatic diisocyanate, a C₂-C₁₂ aliphatic diisothiocyanate, or a combination thereof; or hydroxyl, and x, R¹, R², R⁴, R⁵, R⁶, R⁷, R⁸, R⁹, and R¹⁰ are defined as in aspect 1, except that one occurrence of R¹, R², R⁴, R⁵, R⁶, R⁷, R⁸, R⁹, and R¹⁰ is hydroxyl; provided that the polymerizable composition further comprises a C₂-C₁₂ aliphatic diisocyanate, a C₂-C₁₂ aliphatic diisothiocyanate, or a combination thereof.

Aspect 11: The method of aspect 10, wherein Y is (meth)acryloyloxy; m and n are zero; x is 1; and 1 or 2 occurrences of R¹, R⁵, R⁶, and R¹⁰ are hydroxyl.

Aspect 12: The method of aspect 10, wherein the polymerizable azobenzene monomer has the structure

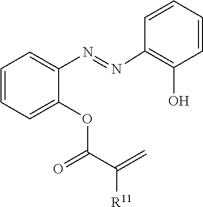

wherein R¹¹ is hydrogen or methyl.

Aspect 13: The method of aspect 1, wherein the dentate mammal or dentate non-mammalian vertebrate comprises exposed dentin in addition to exposed pulp; wherein the method further comprises applying a dental adhesive to the exposed dentin and curing the dental adhesive, prior to the applying a polymerizable composition to exposed dental pulp; wherein the method further comprises applying the polymerizable composition to the cured dental adhesive simultaneously with the applying the polymerizable composition to the exposed dental pulp, and, optionally, air-drying and/or polymerizing the polymerizable composition; wherein the method further comprises applying a restorative composition to the cured dental adhesive and the polymerizable composition, and polymerizing the restorative composition; wherein the restorative composition comprises 50 to 95 weight percent of a particulate inorganic material, and 5 to 50 weight percent of a di(meth)acrylate monomer, based on the total weight of the restorative composition; wherein the polymerizable composition further comprises a solvent selected from the group consisting of C₁-C₆ alkanols, C₃-C₆ ketones, and combinations thereof; and wherein the hydroxy-substituted azobenzene group is a pendant group of a polymerizable azobenzene monomer having the structure

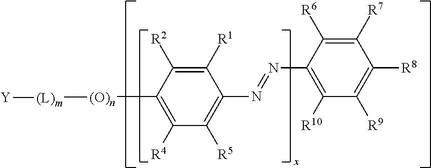

wherein m is zero or 1, and n is zero or 1, provided that if m is zero, then n is zero; L is C₂-C₁₂ alkylene or C₆-C₁₂ arylene; and Y is (meth)acryloyloxy, and x, R¹, R², R⁴, R⁵, R⁶, R⁷, R⁸, R⁹, and R¹⁰ are defined as in aspect 1; or (meth)acrylamido, and x, R¹, R², R⁴, R⁵, R⁶, R⁷, R⁸, R⁹, and R¹⁰ are defined as in aspect 1.

Aspect 14: The method of aspect 13, wherein the polymerizable composition comprises, based on the weight of the polymerizable composition, 0.1 to 5 weight percent of the polymerizable azobenzene monomer, and 10 to 99.9 weight percent of the solvent.

Aspect 15: The method of aspect 14, wherein the polymerizable composition further comprises, based on the weight of the polymerizable composition, 5 to 89.9 weight percent of a (meth)acrylate monomer copolymerizable with the polymerizable monomer.

Aspect 16: The method of aspect 1, wherein the dentate mammal or dentate non-mammalian vertebrate comprises exposed dentin in addition to exposed pulp; wherein the method further comprises applying a dental adhesive to the exposed dentin and curing the dental adhesive, after the applying a polymerizable composition to exposed dental pulp; wherein the method further comprises, optionally, air-drying and/or polymerizing the polymerizable composition prior to the applying a dental adhesive to the exposed dentin and curing the dental adhesive; wherein the method further comprises applying a restorative composition to the cured dental adhesive and the polymerizable composition, and polymerizing the restorative composition; wherein the restorative composition comprises 50 to 95 weight percent of a particulate inorganic material, and 5 to 50 weight percent of a di(meth)acrylate monomer, based on the total weight of the restorative composition; wherein the polymerizable composition further comprises a solvent selected from the group consisting of C₁-C₆ alkanols, C₃-C₆ ketones, and combinations thereof; and wherein the hydroxy-substituted azobenzene group is a pendant group of a polymerizable azobenzene monomer having the structure

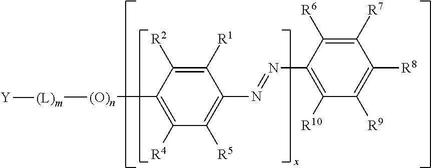

wherein m is zero or 1, and n is zero or 1, provided that if m is zero, then n is zero; L is C₂-C₁₂ alkylene or C₆-C₁₂ arylene; and Y is (meth)acryloyloxy, and x, R¹, R², R⁴, R⁵, R⁶, R⁷, R⁸, R⁹, and R¹⁰ are defined as in aspect 1; or (meth)acrylamido, and x, R¹, R², R⁴, R⁵, R⁶, R⁷, R⁸, R⁹, and R¹⁰ are defined as in aspect 1.

Aspect 17: The method of aspect 16, wherein the polymerizable composition comprises, based on the weight of the polymerizable composition, 0.1 to 5 weight percent of the polymerizable azobenzene monomer, and 10 to 99.9 weight percent of the solvent.

Aspect 18: The method of aspect 16, wherein the polymerizable composition further comprises, based on the weight of the polymerizable composition, 5 to 89.9 weight percent of a (meth)acrylate monomer copolymerizable with the polymerizable monomer.

Aspect 19: The method of aspect 1, wherein the dentate mammal or dentate non-mammalian vertebrate comprises exposed dentin in addition to exposed pulp; wherein the method further comprises applying a dental adhesive to the exposed dentin and curing the dental adhesive prior to the applying a polymerizable composition to exposed dental pulp; wherein the method further comprises applying the polymerizable composition to the cured dental adhesive simultaneously with the applying the polymerizable composition to the exposed dental pulp; and wherein the hydroxy-substituted azobenzene group is a pendant group of a polymerizable azobenzene monomer having the structure

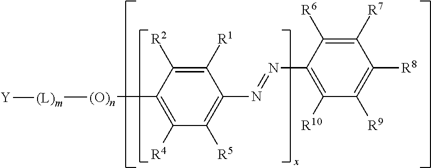

wherein m is zero or 1, and n is zero or 1, provided that if m is zero, then n is zero; L is C₂-C₁₂ alkylene or C₆-C₁₂ arylene; and Y is (meth)acryloyloxy, and x, R¹, R², R⁴, R⁵, R⁶, R⁷, R⁸, R⁹, and R¹⁰ are defined as in aspect 1; or (meth)acrylamido, and x, R¹, R², R⁴, R⁵, R⁶, R⁷, R⁸, R⁹, and R¹⁰ are defined as in aspect 1; and wherein the polymerizable composition is a restorative composition comprising the polymerizable azobenzene monomer, a particulate inorganic filler, and a (meth)acrylate monomer copolymerizable with the polymerizable monomer.

Aspect 20: The method of aspect 19, wherein the restorative composition comprises, based on the total weight of the restorative composition, 0.1 to 5 weight percent of the polymerizable azobenzene monomer, 50 to 94.9 weight percent of the particulate inorganic material, and 5 to 50 weight percent of the (meth)acrylate monomer copolymerizable with the polymerizable azobenzene monomer.

The invention is further illustrated by the following non-limiting examples.

### EXAMPLES

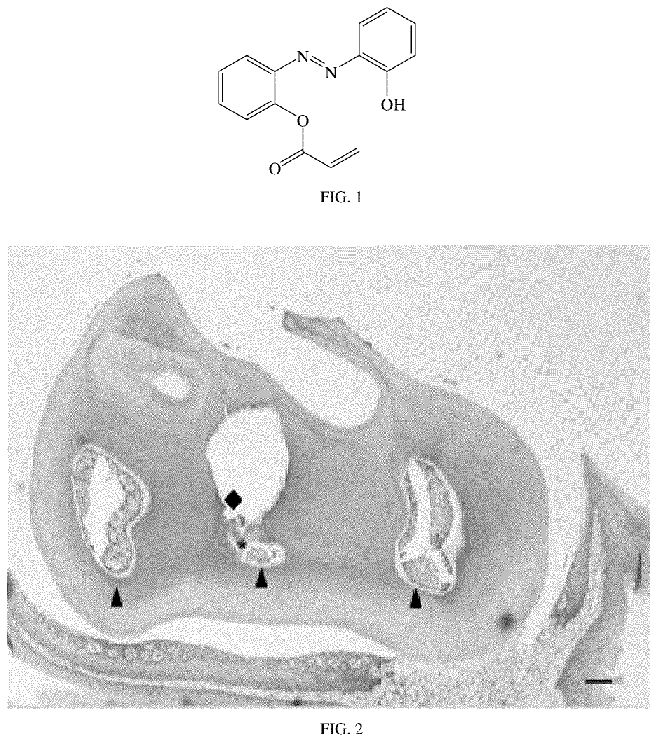

2-Acryloyloxy-2′-hydroxy-azobenzene (Acrylated HydroxyAzobenzene, AHA) was prepared as described in International Patent Application Publication No. WO 2020/172632 A1 of D. Nair et al. The chemical structure of AHA is shown in FIG. 1.

While not wishing to be bound by any particular theory of operation, the present inventors hypothesized that when a hydroxy-substituted azobenzene material, such as AHA or its polymerization product, is in contact with pulp tissue, it can stimulate the native stem cell population to proliferate and form odontoblast-like cells that are formative cells for repair and regeneration of dentin.

In order to test this hypothesis, a class I cavity extending into the dentin and almost to the pulp was prepared on the occlusal surface of the maxillary first molar after anesthesia in five to six week old C57BL/6J mice. The cavity was prepared using a 0.3-0.5 millimeter diameter carbide bur. An endodontic hand file with a tip diameter of 0.06-0.10 millimeter was used to create a mechanical break in the thin dentin overlying the dental pulp, thereby creating a cavity that included exposed dentin and exposed pulp.

For the control group of mice, after hemostasis, a one-step, dual-cure, self-etching bonding agent (SCOTCHBOND™ Universal Adhesive; 3M ESPE, St. Paul, Minnesota, USA) was applied to exposed dentin according to the manufacturer's instructions and cured for 20-40 seconds using a dental curing light operated at 420-480 nanometers and 1200-2000 milliwatts/centimeter². ProRoot™ MTA (Mineral Trioxide Aggregate) root canal repair material (Dentsply Sirona, Tulsa Dental Specialties, Johnson City, Tennessee, USA) was prepared according to manufacturer's instructions by mixing powder and liquid components in equal proportions to form a dough. The dough was packed over the exposed pulp according to manufacturer's instructions. The cavity was then sealed with FILTEK™ Supreme Ultra Universal Restorative (3M ESPE, St. Paul, Minnesota, USA), which was applied to the cured adhesive and the root canal repair material, then cured for 20-40 seconds using a dental curing light operated at 420-480 nanometers and 1200-2000 milliwatts/centimeter².

For the test group of mice, after hemostasis, a one-step, dual-cure, self-etching bonding agent (SCOTCHBOND™ Universal Adhesive; 3M ESPE, St. Paul, Minnesota, USA) was applied to exposed dentin according to the manufacturer's instructions and cured for 20-40 seconds using a dental curing light operated at 420-480 nanometers and 1200-2000 milliwatts/centimeter². Prior to being applied, the AHA solution was divided into two parts of equal volume to mix in the photoinitiators and the redox (self-cure) initiator components. In Part 1, Benzoyl Peroxide (BPO) at 1 mg/mL is added. To Part 2, camphorquinone (CQ) at 0.5 mg/mL, Ethyl-4-(dimethyl-amino)benzoate (A) at 0.5 mg/mL and N,N-dimethyl-p-toluidine (3-A) at 0.5 mg/mL are added. Just before the AHA solution is to applied, Part 1 and Part 2 are mixed together. Exposed dental pulp was then covered with a thin layer (0.1-0.5 microliter) of a 25 milligram/milliliter solution of AHA in a 70:30 weight/weight mixture of ethanol and acetone. The amount of AHA delivered was therefore 2.5 to 12.5 micrograms per restoration (depending on the tooth size). The AHA solution was applied using a 0.5 millimeter microbrush. The applied AHA solution was allowed to air-dry for 3-5 seconds. FILTEK™ Supreme Ultra Universal Restorative was then applied to the AHA layer and the cured bonding agent. The AHA layer and FILTEK™ Supreme Ultra Universal Restorative were then light-cured for 40 seconds using a dental curing light operating at 420-480 nanometer and 1200-2000 milliwatts/centimeter². According to the manufacturer's Material Safety Data Sheet, FILTEK™ Supreme Ultra Universal Restorative includes bisphenol A diglycidyl ether dimethacrylate (BISGMA), bisphenol A polyethylene glycol diether dimethacrylate (BISEMA-6), diurethane dimethacrylate (UDMA), polyethylene glycol dimethacrylate (PEGDMA), and triethylene glycol dimethacrylate, each of which is capable of copolymerizing with AHA.

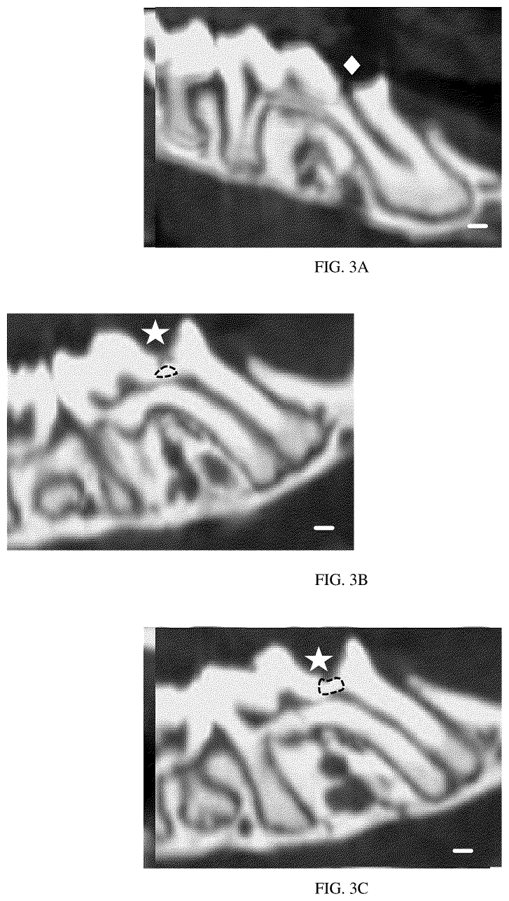

The control and test groups of mice were maintained for four or six weeks following creation and treatment of the cavity, after which they were sacrificed. For mice sacrificed at four weeks, maxilla were harvested from each mouse, dissected, fixed in 4% paraformaldehyde overnight at 4° C., and decalcified in 14% EDTA for 7-10 days. Decalcified tissues were placed in a 30% aqueous sucrose solution overnight and embedded in Shandon CRYOMATRIX™ embedding resin (Thermo Fisher Scientific, Waltham, Massachusetts, USA). Seven-micrometer sections were obtained using a Leica cryostat and mounted using a CryoJane™ Tape Transfer System (Leica Biosystems, Buffalo Grove, Illinois, USA). All the sections were stained with Hematoxylin and Eosin (H&E) to evaluate the formation of dentinal bridge at the site of tissue injury. Sections were examined and imaged using a Zeiss Axioplan II microscope. Microscopy demonstrated vital pulp without any evidence of chronic or acute inflammation or dysplastic changes (FIG. 2, black arrows). The gingiva around the site of application of AHA appeared healthy without any signs of inflammation or dysplasia. The site of pulp exposure (FIG. 2, black diamond) had been closed by formation of a dentinal bridge generated by reparative dentin formation (FIG. 2, black asterisk). In FIG. 2, the black arrows indicate vital pulp, and the scale bar corresponds to 100 micrometers. This experiment demonstrates the promotion of dentin generation by AHA.

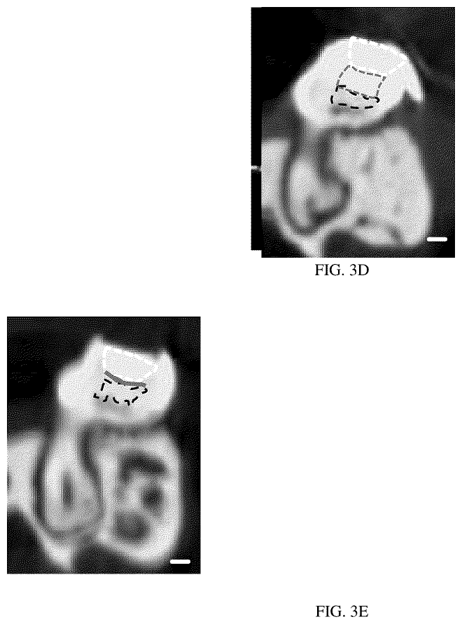

FIG. 3 consists of five images (3A-3E) of maxilla harvested from mice at zero (3A) or six weeks (3B-3E), the images obtained by micro computed tomography (microCT) at the Animal Imaging Core Facility at the University of Colorado. Due to the inherent contrast between bone, air, and tissue structures and the resulting attenuation of the x-rays passing through dense material, microCT is particularly well suited for providing high quality resolution of structure in the bone and teeth, both in vivo and ex vivo. microCT scanners are capable of providing volumetric computed tomography analysis with an isotropic voxel spacing less than 100 micrometers. See, e.g., N. J. Serkova et al., “Preclinical Applications of Multi-Platform Imaging in Animal Models of Cancer,” Cancer Research 2021, volume 81, number 5, pages 1189-1200 (PMID 33262127).

The high-resolution, three-dimensional CT scans were acquired with a PerkinElmer Quantum GX2 microCT Imaging System. A high resolution/high dose CT scan was acquired with the X-ray source set to 88 milliamps current, 90 kilovoltage peak, and 0.06 millimeter copper plus 0.5 millimeter aluminum filters. The scan was obtained in bone mode with a field of view of 18 millimeter², a voxel size of 36 microns (with sub-volume reconstructions), and total scan time of 57 minutes. The reconstructed 3D-datasets, prepared using PerkinElmer AccuCT and Analyze Advanced μCT software, were used to detect the formation of a calcified dentinal bridge in the tooth, and to detect any infection in the bone that may be due to the death of the dental pulp.

The results of microCT analysis are shown in FIG. 3.

FIG. 3A is a longitudinal section of a murine maxillary first molar harvested after the creation of the cavity but before treatment. The white diamond is situated just above the area of exposed pulp; the scale bar in the lower right corner corresponds to 100 micrometers.

FIG. 3B is a longitudinal section of a murine maxillary first molar harvested from a control group mouse six weeks after the dental procedure. Cured restorative was removed from the tooth prior to imaging. The white star indicates the area from which cured restorative was removed. The area surrounded by a black dashed line indicates a pulp exposure site closed by a calcified dentinal bridge. The scale bar in the lower right corner corresponds to 100 micrometers.

FIG. 3C is a longitudinal section of a murine maxillary first molar harvested from a test group mouse six weeks after the dental procedure. Cured restorative was removed from the tooth prior to imaging. The white star indicates the area from which cured restorative was removed. The area surrounded by a black dashed line indicates a pulp exposure site closed by a calcified dentinal bridge. The scale bar in the lower right corner corresponds to 100 micrometers.

FIG. 3D is a coronal section of the same control group molar imaged in FIG. 3B. The area surrounded by a long dashed black line indicates an area of calcified dentin formation. The area surrounded by a short dashed gray line indicates an area of MTA deposition. And the area surrounded by a short dashed white line indicates an area of cured restorative. The scale bar in the lower right corner corresponds to 100 micrometers.

FIG. 3E is a coronal section of the same control group molar imaged in FIG. 3C. The area surrounded by a long dashed black line indicates an area of calcified dentin formation. The area indicated by a solid gray line is an area of AHA deposition. And the area surrounded by a short dashed white line indicates an area of cured restorative. The scale bar in the lower right corner corresponds to 100 micrometers.

1. A method of stimulating dentin formation, comprising:
applying a polymerizable composition to exposed dental pulp in a dentate mammal or a dentate non-mammalian vertebrate; wherein the polymerizable composition comprises a hydroxy-substituted azobenzene group having the structure 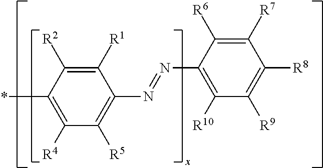
wherein
x is 1, 2, 3, 4, 5, 6, 7, 8, 9, or 10; and
each occurrence of R¹, R², R⁴, R⁵, R⁶, R⁷, R⁸, R⁹, and R¹⁰ is independently hydrogen, methyl, or hydroxyl, provided that at least one occurrence of R¹, R², R⁴, R⁵, R⁶, R⁷, R⁸, R⁹, and R¹⁰ is hydroxyl; and
polymerizing the polymerizable composition;
wherein the amount of the hydroxy-substituted azobenzene group applied to the exposed dental pulp is effective to promote dentin generation.

2. The method of claim 1, wherein, in the hydroxy-substituted azobenzene group, x is 1, and 1 or 2 occurrences of R¹, R⁵, R⁶, and R¹⁰ are hydroxyl.

3. The method of claim 1, wherein the hydroxy-substituted azobenzene group has the structure

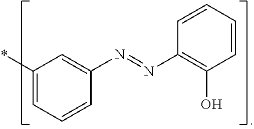

4. The method of claim 1, wherein the applying a polymerizable composition to exposed dental pulp comprises applying the polymerizable composition to exposed dental pulp in an amount effective to provide 0.5 to 50 micrograms of the hydroxy-substituted azobenzene group.

5. The method of claim 1, wherein the applying a polymerizable composition to exposed dental pulp comprises applying the polymerizable composition to exposed dental pulp in an amount effective to provide 5 to 500 micrograms of the hydroxy-substituted azobenzene group per millimeter-squared of the exposed dental pulp.

6. The method of claim 1, wherein the applying a polymerizable composition to exposed dental pulp comprises applying the polymerizable composition to exposed dental pulp in an amount effective to provide 20 to 3000 micrograms of the hydroxy-substituted azobenzene group per kilogram of body weight of the dentate mammal or a dentate non-mammalian vertebrate.

7. The method of claim 1, wherein the hydroxy-substituted azobenzene group is part of a polymerizable azobenzene monomer having the structure 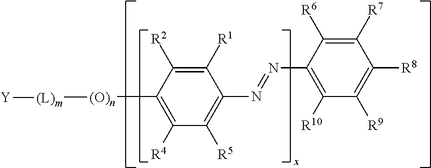
wherein
m is zero or 1, and n is zero or 1, provided that if m is zero, then n is zero;
L is C₂-C₁₂ alkylene or C₆-C₁₂ arylene; and
Y is
(meth)acryloyloxy, and x, R¹, R², R⁴, R⁵, R⁶, R⁷, R⁸, R⁹, and R¹⁰ are defined as in claim 1; or
(meth)acrylamido, and x, R¹, R², R⁴, R⁵, R⁶, R⁷, R⁸, R⁹, and R¹⁰ are defined as in claim 1; or
isocyanato, and x, R¹, R², R⁴, R⁵, R⁶, R⁷, R⁸, R⁹, and R¹⁰ are defined as in claim 1, except that one occurrence of R¹, R², R⁴, R⁵, R⁶, R⁷, R⁸, R⁹, and R¹⁰ is isocyanato; provided that the polymerizable composition further comprises a C₂-C₁₂ aliphatic diol, a C₂-C₁₂ aliphatic dithiol, or a combination thereof; or
isothiocyanato, and x, R¹, R², R⁴, R⁵, R⁶, R⁷, R⁸, R⁹, and R¹⁰ are defined as in claim 1, except that one occurrence of R¹, R², R⁴, R⁵, R⁶, R⁷, R⁸, R⁹, and R¹⁰ is isothiocyanato; provided that the polymerizable composition further comprises a C₂-C₁₂ aliphatic diol, a C₂-C₁₂ aliphatic dithiol, or a combination thereof; or
carboxyl, and x, R¹, R², R⁴, R⁵, R⁶, R⁷, R⁸, R⁹, and R¹⁰ are defined as in claim 1, except that one occurrence of R¹, R², R⁴, R⁵, R⁶, R⁷, R⁸, R⁹, and R¹⁰ is carboxyl; provided that the polymerizable composition further comprises a C₂-C₁₂ aliphatic diol, a C₂-C₁₂ aliphatic diamine, or a combination thereof; or
thiol, and x, R¹, R², R⁴, R⁵, R⁶, R⁷, R⁸, R⁹, and R¹⁰ are defined as in claim 1, except that one occurrence of R¹, R², R⁴, R⁵, R⁶, R⁷, R⁸, R⁹, and R¹⁰ is thiol; provided that the polymerizable composition further comprises a C₂-C₁₂ aliphatic diisocyanate, a C₂-C₁₂ aliphatic diisothiocyanate, or a combination thereof; or
hydroxyl, and x, R¹, R², R⁴, R⁵, R⁶, R⁷, R⁸, R⁹, and R¹⁰ are defined as in claim 1, except that one occurrence of R¹, R², R⁴, R⁵, R⁶, R⁷, R⁸, R⁹, and R¹⁰ is hydroxyl; provided that the polymerizable composition further comprises a C₂-C₁₂ aliphatic diisocyanate, a C₂-C₁₂ aliphatic diisothiocyanate, or a combination thereof.

8. The method of claim 7, wherein, in the polymerizable azobenzene monomer, Y is (meth)acryloyloxy; m and n are zero; x is 1; and 1 or 2 occurrences of R¹, R⁵, R⁶, and R¹⁰ are hydroxyl.

9. The method of claim 7, wherein the polymerizable azobenzene monomer has the 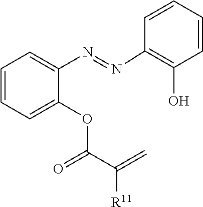
wherein R¹¹ is hydrogen or methyl.

10. The method of claim 1, wherein
the polymerizable composition further comprises a curable monomer; and
the hydroxy-substituted azobenzene group is a pendant group of a polymer comprising the residue of a polymerizable azobenzene monomer having the structure 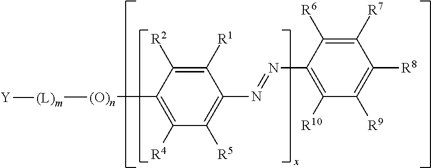
wherein
m is zero or 1, and n is zero or 1, provided that if m is zero, then n is zero;
L is C₂-C₁₂ alkylene or C₆-C₁₂ arylene; and
Y is
(meth)acryloyloxy, and x, R¹, R², R⁴, R⁵, R⁶, R⁷, R⁸, R⁹, and R¹⁰ are defined as in claim 1; or
(meth)acrylamido, and x, R¹, R², R⁴, R⁵, R⁶, R⁷, R⁸, R⁹, and R¹⁰ are defined as in claim 1; or
isocyanato, and x, R¹, R², R⁴, R⁵, R⁶, R⁷, R⁸, R⁹, and R¹⁰ are defined as in claim 1, except that one occurrence of R¹, R², R⁴, R⁵, R⁶, R⁷, R⁸, R⁹, and R¹⁰ is isocyanato; provided that the polymerizable composition further comprises a C₂-C₁₂ aliphatic diol, a C₂-C₁₂ aliphatic dithiol, or a combination thereof; or
isothiocyanato, and x, R¹, R², R⁴, R⁵, R⁶, R⁷, R⁸, R⁹, and R¹⁰ are defined as in claim 1, except that one occurrence of R¹, R², R⁴, R⁵, R⁶, R⁷, R⁸, R⁹, and R¹⁰ is isothiocyanato; provided that the polymerizable composition further comprises a C₂-C₁₂ aliphatic diol, a C₂-C₁₂ aliphatic dithiol, or a combination thereof; or
carboxyl, and x, R¹, R², R⁴, R⁵, R⁶, R⁷, R⁸, R⁹, and R¹⁰ are defined as in claim 1, except that one occurrence of R¹, R², R⁴, R⁵, R⁶, R⁷, R⁸, R⁹, and R¹⁰ is carboxyl; provided that the polymerizable composition further comprises a C₂-C₁₂ aliphatic diol, a C₂-C₁₂ aliphatic diamine, or a combination thereof; or
thiol, and x, R¹, R², R⁴, R⁵, R⁶, R⁷, R⁸, R⁹, and R¹⁰ are defined as in claim 1, except that one occurrence of R¹, R², R⁴, R⁵, R⁶, R⁷, R⁸, R⁹, and R¹⁰ is thiol; provided that the polymerizable composition further comprises a C₂-C₁₂ aliphatic diisocyanate, a C₂-C₁₂ aliphatic diisothiocyanate, or a combination thereof; or
hydroxyl, and x, R¹, R², R⁴, R⁵, R⁶, R⁷, R⁸, R⁹, and R¹⁰ are defined as in claim 1, except that one occurrence of R¹, R², R⁴, R⁵, R⁶, R⁷, R⁸, R⁹, and R¹⁰ is hydroxyl; provided that the polymerizable composition further comprises a C₂-C₁₂ aliphatic diisocyanate, a C₂-C₁₂ aliphatic diisothiocyanate, or a combination thereof.

11. The method of claim 10, wherein Y is (meth)acryloyloxy; m and n are zero; x is 1; and 1 or 2 occurrences of R¹, R⁵, R⁶, and R¹⁰ are hydroxyl.

12. The method of claim 10, wherein the polymerizable azobenzene monomer has the structure 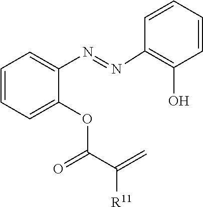
wherein R¹¹ is hydrogen or methyl.

13. The method of claim 1,
wherein the dentate mammal or dentate non-mammalian vertebrate comprises exposed dentin in addition to exposed pulp;
wherein the method further comprises applying a dental adhesive to the exposed dentin and curing the dental adhesive, prior to the applying a polymerizable composition to exposed dental pulp;
wherein the method further comprises applying the polymerizable composition to the cured dental adhesive simultaneously with the applying the polymerizable composition to the exposed dental pulp, and, optionally, air-drying and/or polymerizing the polymerizable composition;
wherein the method further comprises applying a restorative composition to the cured dental adhesive and the polymerizable composition, and polymerizing the restorative composition; wherein the restorative composition comprises 50 to 95 weight percent of a particulate inorganic material, and 5 to 50 weight percent of a di(meth)acrylate monomer, based on the total weight of the restorative composition
wherein the polymerizable composition further comprises a solvent selected from the group consisting of C₁-C₆ alkanols, C₃-C₆ ketones, and combinations thereof; and
wherein the hydroxy-substituted azobenzene group is a pendant group of a polymerizable azobenzene monomer having the structure 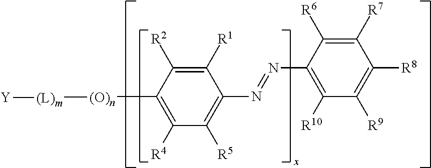
wherein
m is zero or 1, and n is zero or 1, provided that if m is zero, then n is zero;
L is C₂-C₁₂ alkylene or C₆-C₁₂ arylene; and
Y is (meth)acryloyloxy, and x, R¹, R², R⁴, R⁵, R⁶, R⁷, R⁸, R⁹, and R¹⁰ are defined as in claim 1; or (meth)acrylamido, and x, R¹, R², R⁴, R⁵, R⁶, R⁷, R⁸, R⁹, and R¹⁰ are defined as in claim 1.

14. The method of claim 13, wherein the polymerizable composition comprises, based on the weight of the polymerizable composition, 0.1 to 5 weight percent of the polymerizable azobenzene monomer, and 10 to 99.9 weight percent of the solvent.

15. The method of claim 14, wherein the polymerizable composition further comprises, based on the weight of the polymerizable composition, 5 to 89.9 weight percent of a (meth)acrylate monomer copolymerizable with the polymerizable monomer.

16. The method of claim 1,
wherein the dentate mammal or dentate non-mammalian vertebrate comprises exposed dentin in addition to exposed pulp;
wherein the method further comprises applying a dental adhesive to the exposed dentin and curing the dental adhesive, after the applying a polymerizable composition to exposed dental pulp;
wherein the method further comprises, optionally, air-drying and/or polymerizing the polymerizable composition prior to the applying a dental adhesive to the exposed dentin and curing the dental adhesive;
wherein the method further comprises applying a restorative composition to the cured dental adhesive and the polymerizable composition, and polymerizing the restorative composition; wherein the restorative composition comprises 50 to 95 weight percent of a particulate inorganic material, and 5 to 50 weight percent of a di(meth)acrylate monomer, based on the total weight of the restorative composition;
wherein the polymerizable composition further comprises a solvent selected from the group consisting of C₁-C₆ alkanols, C₃-C₆ ketones, and combinations thereof; and
wherein the hydroxy-substituted azobenzene group is a pendant group of a polymerizable azobenzene monomer having the structure 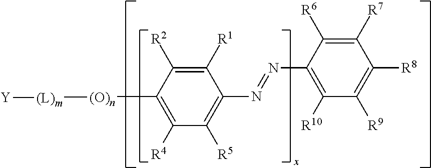
wherein
m is zero or 1, and n is zero or 1, provided that if m is zero, then n is zero;
L is C₂-C₁₂ alkylene or C₆-C₁₂ arylene; and
Y is (meth)acryloyloxy, and x, R¹, R², R⁴, R⁵, R⁶, R⁷, R⁸, R⁹, and R¹⁰ are defined as in claim 1; or (meth)acrylamido, and x, R¹, R², R⁴, R⁵, R⁶, R⁷, R⁸, R⁹, and R¹⁰ are defined as in claim 1.

17. The method of claim 16, wherein the polymerizable composition comprises, based on the weight of the polymerizable composition, 0.1 to 5 weight percent of the polymerizable azobenzene monomer, and 10 to 99.9 weight percent of the solvent.

18. The method of claim 16, wherein the polymerizable composition further comprises, based on the weight of the polymerizable composition, 5 to 89.9 weight percent of a (meth)acrylate monomer copolymerizable with the polymerizable monomer.

19. The method of claim 1,
wherein the dentate mammal or dentate non-mammalian vertebrate comprises exposed dentin in addition to exposed pulp;
wherein the method further comprises applying a dental adhesive to the exposed dentin and curing the dental adhesive prior to the applying a polymerizable composition to exposed dental pulp;
wherein the method further comprises applying the polymerizable composition to the cured dental adhesive simultaneously with the applying the polymerizable composition to the exposed dental pulp; and
wherein the hydroxy-substituted azobenzene group is a pendant group of a polymerizable azobenzene monomer having the structure 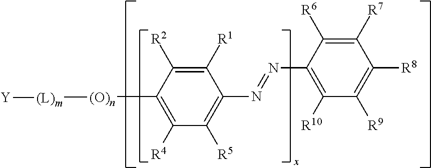
wherein
m is zero or 1, and n is zero or 1, provided that if m is zero, then n is zero;
L is C₂-C₁₂ alkylene or C₆-C₁₂ arylene; and
Y is (meth)acryloyloxy, and x, R¹, R², R⁴, R⁵, R⁶, R⁷, R⁸, R⁹, and R¹⁰ are defined as in claim 1; or (meth)acrylamido, and x, R¹, R², R⁴, R⁵, R⁶, R⁷, R⁸, R⁹, and R¹⁰ are defined as in claim 1; and
wherein the polymerizable composition is a restorative composition comprising the polymerizable azobenzene monomer, a particulate inorganic filler, and a (meth)acrylate monomer copolymerizable with the polymerizable monomer.

20. The method of claim 19, wherein the restorative composition comprises, based on the total weight of the restorative composition, 0.1 to 5 weight percent of the polymerizable azobenzene monomer, 50 to 94.9 weight percent of the particulate inorganic material, and 5 to 50 weight percent of the (meth)acrylate monomer copolymerizable with the polymerizable azobenzene monomer.
